(19) United States
(12) Patent Application Publication
(10) Pub. No.: US 2025/0276109 A1
(43) Pub. Date: Sep. 4, 2025

(54) SEMI-SYNTHETIC SCAFFOLD FOR WOUND HEALING & TISSUE ENGINEERING

(71) Applicant: Asclepii Inc., Cleveland, OH (US)

(72) Inventors: Franco Kraiselburd, Cleveland, OH (US); Ixchel Airi Robles Ruiz, Cuernavaca, Morelos (MX); Miguel Angel Fuentes Chandia, Cleveland Heights, OH (US); Daniel Katzman, Newton, MA (US); Santiago Kraiselburd Monti, Monterrey Centro (MX); Mahshid Monavari, Cleveland Heights, OH (US)

(21) Appl. No.: 19/066,431

(22) Filed: Feb. 28, 2025

Related U.S. Application Data
(60) Provisional application No. 63/559,449, filed on Feb. 29, 2024.

(51) Int. Cl. A61L 27/22; A61L 27/20; A61L 27/54; A61L 27/56

(52) U.S. Cl. CPC A61L 27/222; A61L 27/20; A61L 27/54; A61L 27/56

(57) ABSTRACT

A method of making a semi-synthetic scaffold is described. The method includes mixing a gelatin solution, a high molecular weight chitosan solution, and a hyaluronate solution, to form a polymeric liquid solution; pouring the polymeric liquid solution into a mold; gradually cooling the mixture to gellify and form a polymer matrix; and freezing and lyophilizing the polymer matrix to remove water and form a scaffold. Semi-synthetic scaffolds, and methods of using them for wound healing, are also described.

### 1. CROSS-REFERENCE TO RELATED APPLICATIONS

This application claims priority to U.S. Provisional Patent Application Ser. No. 63/559,449, filed Feb. 29, 2024, which is incorporated herein by reference.

### 2. BACKGROUND

Chronic wounds are one of the most uneven and pressing global crises, with over 11 million people suffering from amputation or death as a direct result from an improperly healed wound. Wounds can result in the skin being disrupted by trauma (e.g. laceration, abrasion, burns or puncture) or by ulceration (e.g. diabetic foot ulcers). Disruptions in the skin that do not heal, or spontaneously recur, are known as chronic wounds. These wounds can take from 12-16 weeks to heal, and cost from $30K-45K per wound due to lack of affordable advanced care. There is a paucity for scalable, cost-effective advanced solutions for wounds. Hemostasis, inflammation, proliferation, and tissue remodeling are the four basic processes of wound healing. Generally, acute wounds heal faster and with fewer complications than chronic wounds. Upon injury, blood vessels contract to deter blood leakage, followed by primary and secondary hemostasis. In contrast, chronic wounds will similarly begin the healing process, but will have prolonged inflammatory, proliferative, or remodeling phases, resulting in tissue fibrosis and in non-healing ulcers. The process of wound healing is complex and involves a variety of specialized cells, such as platelets, macrophages, fibroblasts, epithelial and endothelial cells. Rodrigues et al., Physiol Rev., 99(1):665-706 (2019). These cells interact with each other and with the extracellular matrix. In addition to the various cellular interactions, healing is also influenced by the action of proteins and glycoproteins, such as cytokines, chemokines, growth factors, inhibitors, and their receptors. Each stage of wound healing has certain milestones that must occur in order for normal healing to progress. If these milestones are not met, a wound will not heal and will instead remain stagnant. These wounds are referred to as “chronic wounds”. Chronic wounds are most likely to occur in limbs and in individuals with certain predispositions such as diabetes, vascular deficiencies, obesity, genetic predispositions, cancer, hypertension, or other. Diabetic patients often encounter a higher rate of occurrence of chronic wounds in the legs or feet due to relatively lower blood flow and skin dryness, which in turn leads to a higher likelihood of skin ulceration.

Approximately 1% to 2% of individuals will be affected by chronic ulceration during their lifetime, and this figure will likely increase as the population ages (Rees and Hirshberg, Adv Wound Care 1999; 12:4-7 (1999). In the United States alone, chronic wounds affect more than 6 million people a year, with increasing numbers anticipated as the population ages. Powers et al., J Am Acad Dermatol., 74(4):607-25 (2016). Patient groups suffering chronic wounds include, but are not limited to, diabetic patients, geriatric patients and patients with circulatory problems. Chronic wounds can also appear as a result of acute trauma or as a post-surgery symptom. Chronic wounds may vary in size, depth and stage of healing, and can contain necrotic tissue, infection, scabs, or exudates (purulent, cerotic).

Chronic wounds can be classified by their cause such as pressure, diabetic, ischemic, venous, and tear and/or by the nature of the wound itself such as its depth and/or stage of healing and/or discharge and/or infections. Chronic wounds are also deeply affected by and associated with bacterial and/or fungal infections, which keep wounds chronic as infected wounds will not heal. Burns are another wound type which is difficult to treat. Conventional burn treatment typically relies upon a topical antibiotic cream (e.g. silver sulfadiazine) followed by a non-stick dressing and gauze. Use of biologic dressings based upon cultured cell grafts and/or fractionated blood products has also been suggested. According to different burn management strategies, frequency of dressing changes can vary from twice per day to about once per week.

A wound dressing for treating chronic wounds should possess the following functions: (1) provide a barrier to protect the wound from contamination by chemicals and microorganisms; (2) allow air exchange, which provides oxygen for cell growth and excreting carbon dioxide produced by cells; (3) remove wound exudates in time and maintain the moist microenvironment between wound, wound exudates, and dressings; (4) be non-toxic and non-allergenic to wound.

Examples of wound treatment dressings include synthetic and natural dressings. Synthetic polymers used to prepare synthetic wound treatments are advantageous in a few characteristics such as tunable properties, endless forms, and established structures over natural polymers. The support offered by synthetic biomaterials can enable restoration of damaged or diseased tissue structure and function. Polymerization, interlinkage, and functionality (changed by block structures, by combining them, by copolymerization) of their molecular weight, molecular structure, physical and chemical features make them easily synthesized as compared to naturally occurring polymers. However, disadvantages of synthetic biomaterials include that they lack cell adhesion sites (e.g., RDG sequences) and typically exhibit low skin integration.

Natural wound dressings are made from natural skin and include both autographs and allografts. Skin obtained from a small biopsy can be grown in cell culture to provide large amounts of skin tissue within three to four weeks. Natural wound dressings provide very effective skin integration, but are difficult to prepare and handle, and are ill-suited for including chemical and biological supplements.

Functional wound dressings possess various bioactivities, such as rapid hemostasis, antibacterial, anti-inflammatory, promoting cell proliferation, tissue regeneration, and wound healing. Functional wound dressings can be provided in two different ways. The first is to prepare dressings directly from biologically active materials such as collagen, chitosan, and alginate. The second is to add bioactive macromolecules or small molecules into the dressings, such as proteins, nanoparticles, growth factors, drugs, and even cells.

Gelatin-/chitosan-/hyaluronan-based biomaterials are used as scaffolds to provide functional wound dressings. Acevedo et al., Bioprocess Biosyst Eng., 36(3):317-24 (2013). These biomaterials are prepared by mixing gelatin (1%) with chitosan (2% in 1% acetic acid) and hyaluronic acid (0.01%) in proportions of 7:2:1 to provide a scaffold made up of 63.58% of gelatin, 36.33% of chitosan and 0.09% of hyaluronic acid. After cooling in a mold, the scaffold is frozen and lyophilized, and then the matrix is cross-linked. The resultant cross-linked sponge is washed with ethanol and frozen and lyophilized again, producing a stable dry polymer.

Use of this scaffold is described in US Patent Publication No. 20140242181 describes a procedure in which a patient's blood is drawn and inserted into a tube with calcium chloride that can be added to the existing matrix in the operating room before the scaffold is placed on the patient. This procedure is effective, both in-vitro, in-vivo in a rabbit model, and in anecdotal human cases. However, the process takes an additional 1.5 hours in the operating room, consuming valuable time and extra medical expenses. This is because incorporating calcium chloride at the patient-facing step is a lengthy and unnecessary step, in addition to the additional manufacturing time and cost required due to the cross-linking.

Using prior art methods, in order to achieve the appropriate properties for cell growth and scaffolding, the scaffold composition undergoes a gradual cooling over 3 days and a freeze-drying process at −80° C. for an additional 12-24 hours in order to achieve a certain crystallization pattern of the water inside the solution and to create a porous matrix with optimal porosity for cell growth as the water is removed. This process can take approximately 4 days. In the current state of the art, Ge/Ch/HA matrices comprise low molecular weight chitosan and are typically cross-linked with an EDC-NHMS-MES sequence, and require washing, followed by an additional cooling and freeze-drying cycle.

According to the prior art, in order to incorporate a cross-linker onto the matrix, current processes consist of wetting the matrix with a cross-linker for it to be re-frozen, which requires an additional 3 days to manufacture, including duplicate gradual cooling and freeze-drying sequences. Additionally, this process makes it difficult or impossible to incorporate additional compounds into the matrix. Prior art methods apply the cross-linker in liquid format such that it is absorbed into the matrix, which in turn slightly degrades the matrix and releases entrapped compounds, limiting its drug delivery capabilities. In simpler terms, by wetting the matrix with a cross-linker, its “drug”/bioactive retention capacity decreases, which makes it hard for the matrix to act as a drug-delivery platform—a much needed product capability in the broader wound healing and tissue engineering field. Additionally, the prior art cross-linking processes of the matrix limit its bioavailability. For the matrix to integrate with the receiving skin properly, the cells must work harder to break the cross-linked molecular bonds.

Accordingly, there remains a need for effective scaffolds for treating chronic wounds that can be prepared quickly and inexpensively while providing good skin integration. This can further enable more streamlined and accessible cellular therapy (in wound healing and other contexts such as heart and liver transplants) and reducing manufacturing and material costs for biomanufactured organs—an area which has been growing significantly recently and requires advanced and cost-effective scaffolding.

### 3. SUMMARY OF THE INVENTION

The method of the present invention removes several steps that are required in prior art methods of making scaffolds for wound healing and tissue engineering. The present invention includes only a single lyophilization (freeze-drying) step and does not include the cross-linking step.

An object of the present invention is providing a semi-synthetic scaffold for wound healing and tissue engineering. The scaffold comprises a composition of gelatin, chitosan, and hyaluronic acid and optionally one or more additives. This composition can be formed into any shape and size and in one embodiment is used as a primary wound dressing. In another embodiment, it can be used as a scaffold for cell and/or whole blood therapy. In another embodiment, it can be used in a biomanufacturing setting as part of a bioreactor system in order to enhance and enable organ manufacturing. The scaffold is referred to as semi-synthetic, because it provides both the advantages of a synthetic scaffold with regard to its ability to easily incorporate additives, while also exhibiting the high skin biocompatibility due to the integration provided by natural or organic scaffolds.

Another object of the present invention is to eliminate the use of traditional cross-linkers in Gelatin Chitosan Hyaluronic Acid/Hyaluronate scaffolds known in the field. This is accomplished in several ways. One embodiment completely eliminates any cross-linking type component, while still maintaining polymer matrix structure. Other embodiments utilize cross-linker alternatives to add additional structure to the polymer matrix. In both cases, production time and complexity is reduced while producing a scaffold with improved cellular adhesion. Another benefit of incorporating a Calcium-based cross-linker alternative directly into the polymer matrix as opposed to externally in a clinical handling process, avoids unnecessary processing steps in the incorporation process of autologous inserts such as a skin biopsy. A prior art method is shown in FIG. 1, while an embodiment of the present invention is shown in FIG. 2. Using the method of the invention, the bioavailability was increased while maintaining good structural integrity with higher MW chitosan, and eliminating the cross-linker component.

The present method departs from the conventional concepts and designs of the prior art and provides a more efficient scaffold that increases the bioavailability of the components incorporated for instance cells and requires less time and less cost to manufacture.

The present invention provides a number of improvements over the prior art. It reduces the time necessary to prepare a scaffold from the prior art process. It removes the second cooling and freeze-drying steps, reducing time and expense from a complex process. It enables the incorporation of other “less traditional” or “weaker” compounds that work as cross-linking agents such as plasticizers and a calcium partial cross-linker, and incorporates any cross-linker from the beginning, instead of after the initial lyophilization step. Instead of using a cross-linker that requires the double lyophilization step, calcium salts can be used as a partial cross-linker and can be added during the original mixing step in the manufacturing process. More specifically, it is added according to the solubility in water of the calcium compound used.

### BRIEF DESCRIPTION OF THE FIGURES

The present invention may be more readily understood by reference to the following figures, wherein:

FIG. 1 provides a scheme showing a process diagram of the main steps of the manufacturing process described by the prior art.

FIG. 2 provide a scheme showing a process diagram of the manufacturing process of the present invention that does not include a cross-linker. Note the additives can be mixed into any of the main base ingredient solutions depending on the most appropriate solvent for the additive. The process is simpler, eliminating the second cooling and lyophilization steps, and allows for simpler addition of optional additive components. Additionally, it allows for less chemical uncertainty or risk as it only requires mixing at 2 points as opposed to 4.

FIGS. 3A-3C provide graphs and images showing: (A) a detailed SEM image of the matrix of the present invention where silver nanoparticles are imbued onto the matrix. Several point and area analyses were conducted at 150 ppm, 10 ppm and 5 ppm in features that appeared to be nanoparticles; (B)-(C) energy dispersive spectrographs (EDS) of the matrix. The spectrographs denote the chemical makeup. The spectrographs show several peaks of silver of different sizes and molecular weights, copper, iron, zinc, carbon (predominant), and oxygen (predominant). This shows the present invention is able to incorporate silver and disperse the nanoparticles homogeneously and does not agglomerate the nanosilver across the matrix pores. This minimizes toxicity and maximizes antimicrobial activity. This sample does not have Ca added, and no palladium coating was applied in order to more clearly detect silver.

FIGS. 4A-4B provide images showing SEM and EDS analysis of a palladium-coated sample with 0.35 M calcium lactate and 5 ppm silver nanoparticle additives. (A) shows SEM image at 750×, showing the desired pore structure is maintained after integrating the calcium and silver additives. (B) is EDC analysis conducted on the same area, specifically showing the calcium (Ca) presence and distribution.

FIGS. 5A-5B provide graphs showing comparison of elemental makeup in control (A) and a calcium and silver additive combination sample (B). Sample (B) shows increased silver (Ag) relative to the control.

FIG. 6 provides a graph showing cell count over time comparing different samples of the present invention compared to a control. Using a novel biosensor technology, we were able to use luciferase-expressing cells in order to quantify the number of cells present in a dish with different scaffold groups present. Through this method we observed the behaviors of cells that we can modulate through the use of showing different additives.

FIG. 7 provide pore size analysis based on SEM images at a 100 magnification. Over 100 pores were analyzed with different additives and concentrations in order to observe any possible impact of additives on the scaffold's porous 3D structure. Overall, different additive levels did not have a significant impact on the pore size.

FIG. 8 provides a graph showing absorption/swelling capacity of the polymer matrix after 3 days was measured for various levels of additives vs. no additives as the control. Note that the control has very little weight, therefore it can absorb 40 times its weight. The other ones absorb 20 times its weight but relatively start with more weight. Additionally, they absorb less after 3 days because they deliver the additive, acting as a delivery system.

FIGS. 9A-9B provide graphs showing results of WST-1 assay (cell metabolic activity) ran over a period of 7 days on groups with increasing concentrations of silver nanoparticles. Cells were seeded onto the scaffolds and left for one day, after which the scaffolds were taken out in order to determine percent adhesion. These results suggest differences in cell behavior depending on silver concentrations. There is a statistically significant improvement in cell adhesion in 1 ppm compared to no silver. Graph (B) shows absorbance over time, and trendlines for cellular activity. 1 and 5 ppm showed the best cell adhesion results.

FIGS. 10A-10B provide graphs showing results of WST-1 assay (cell metabolic activity) ran over a period of 7 days on groups with increasing concentrations of calcium. Cells were seeded onto the scaffolds and left for one day, after which the scaffolds were taken out in order to determine percent adhesion. These results suggest differences in cell behavior depending on calcium concentrations.

### 4. DETAILED DESCRIPTION OF THE INVENTION

The present invention provides a method of making a semi-synthetic scaffold as described. The method includes mixing a gelatin solution, a high molecular weight chitosan solution, and a hyaluronate solution, to form a polymeric liquid solution; pouring the polymeric liquid solution into a mold; gradually cooling the mixture to a temperature of −80° C. for 12 to 24 hours to form a polymer matrix; and freezing and lyophilizing the polymer matrix to form a scaffold. Semi-synthetic scaffolds, and methods of using them for wound healing, are also provided.

### 5. DEFINITIONS

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The phrase “consisting essentially of” means that the composition or method may include additional ingredients and/or steps, but only if the additional ingredients and/or steps do not materially alter the basic and novel characteristics of the claimed composition or method.

“Pharmaceutically acceptable” as used herein means that the compound or composition is suitable for administration to a subject for the methods described herein, without unduly deleterious side effects in light of the severity of the disease and necessity of the treatment.

“Biocompatible” as used herein, refers to any material that does not cause injury or death to a subject or induce an adverse reaction in a subject when placed in contact with the subject's tissues. Adverse reactions include for example inflammation, infection, and cell death. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

The term “biodegradable” as used herein refers to a polymer that can be broken down by either chemical or physical process, upon interaction with the physiological environment subsequent to administration, and erodes or dissolves within a period of time, typically within days, weeks, or months. A biodegradable material serves a temporary function in the body, and is then degraded or broken into components that are metabolizable or excretable.

The terms “therapeutically effective” and “pharmacologically effective” are intended to qualify the amount of an agent which will achieve the goal of improvement in disease severity and the frequency of incidence.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

“Contacting,” as used herein, refers to causing two items to become physically adjacent and in contact, or placing them in an environment where such contact will occur within a short timeframe. For example, contacting a wound with a semi-synthetic scaffold includes administering the composition to a subject at or near a site such that the scaffold will encourage wound healing. In some embodiments, the step of contacting the site comprises surgically positioning or implanting the composition.

“Cross-linking” is the process of creating chemical bonds between two atoms in a compound. In the context of wound healing and tissue engineering, cross-linking is referred to more specifically as the process of stabilizing collagen or compounds within medical devices by creating new covalent bonds between strands of collagen; this process inhibits degradation of the collagen by proteases and prolongs its presence in the wound.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a compound” also includes a plurality of such compounds.

As used herein, the terms “treatment”, “treating”, and the like, refer to obtaining a desired pharmacologic or physiologic effect. The effect may be therapeutic in terms of a partial or complete cure for a disease or an adverse effect attributable to the disease. “Treatment”, as used herein, covers any treatment of a disease in a mammal, particularly in a human, and can include inhibiting the disease or condition, i.e., arresting its development; and relieving the disease, i.e., causing regression of the disease.

All scientific and technical terms used in the present application have meanings commonly used in the art unless otherwise specified. The definitions provided herein are to facilitate understanding of certain terms used frequently herein and are not meant to limit the scope of the present application.

### 6. METHODS OF MAKING A SEMI-SYNTHETIC SCAFFOLD

One aspect of the invention provides a method of making a semi-synthetic scaffold. The method includes the steps of mixing a gelatin solution, a high molecular weight chitosan solution, and a hyaluronate solution, to form a polymeric liquid solution; pouring the polymeric liquid solution into a mold; gradually cooling the mixture to a temperature of −80° C. for 12 to 24 hours to form a polymer matrix; and freezing and lyophilizing the polymer matrix to form a scaffold. The process for making a semi-synthetic scaffold is illustrated in FIG. 2. The gelatin, high molecular weight chitosan, and hyaluronate mixture is also referred to herein as Ge/Ch/HA.

The purpose of cross-linking is to increase the shelf-life of the matrix and create a stronger, more robust scaffolding for mid-to-long term applications. However, the inventors have observed that chitosan having a higher molecular weight can achieve similar results without the need for a cross-linking agent whatseoever. This marks a significant innovation over the existing production methods, as it decreases costs and manufacturing times, while providing good cellular adhesion for wound healing.

As an alternative embodiment, the inventors have found that eliminating the crosslinker altogether, incorporating less common cross-linker type components in earlier process steps, and using other compounds such as calcium or plasticizers (such as glycerol) produces a suitable polymer matrix. They have observed that incorporating the cross-linkers directly into the product produces a similar matrix without additional processing steps, saving over 3 days in manufacturing steps and allowing for better incorporation of additives (e.g., drugs) in the polymer matrix. While in some embodiments, removal of the cross-linker is beneficial, there are certain applications that can favor a cross-linker (especially in biomanufacturing settings), and a method using a 7-2-1 Ge/Ch/HA composition manufactured with all of the cross-linkers in the solution from the beginning is a marked improvement upon the current state of the art as incorporating the cross-linker from the beginning has been observed by the inventors as a way to make the scaffold in a streamlined, cost-effective manner.

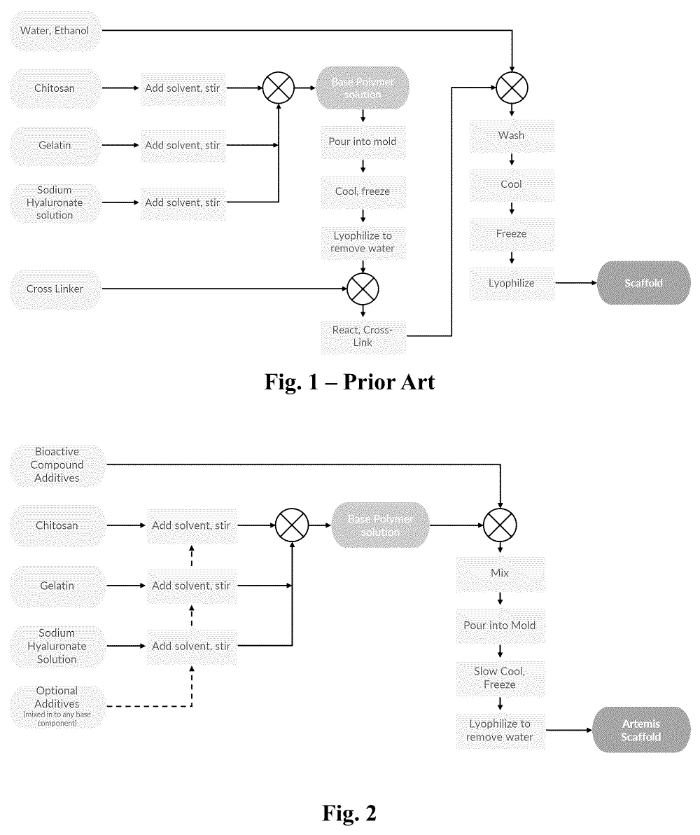

As shown in FIG. 2, one of the differences of the present invention from prior art methods is that the steps of freezing and lyophilizing the polymer matrix are only done once, rather than being done once after mixing, and then again after cross-linking. Another difference between the method and methods used in the prior art is that a crosslinking agent does not need to be added after freezing and lyophilizing the polymer matrix. The prior art method shown in FIG. 1, on the other hand, includes the step of adding a crosslinking agent after the first round of freezing and lyophilizing the polymer matrix. Several embodiments that eliminate the secondary freeze and freeze-drying steps are described herein. In the present disclosure, both embodiments that do not contain any type of cross-linker and compounds that function similarly to crosslinkers are made without the secondary freeze-drying steps.

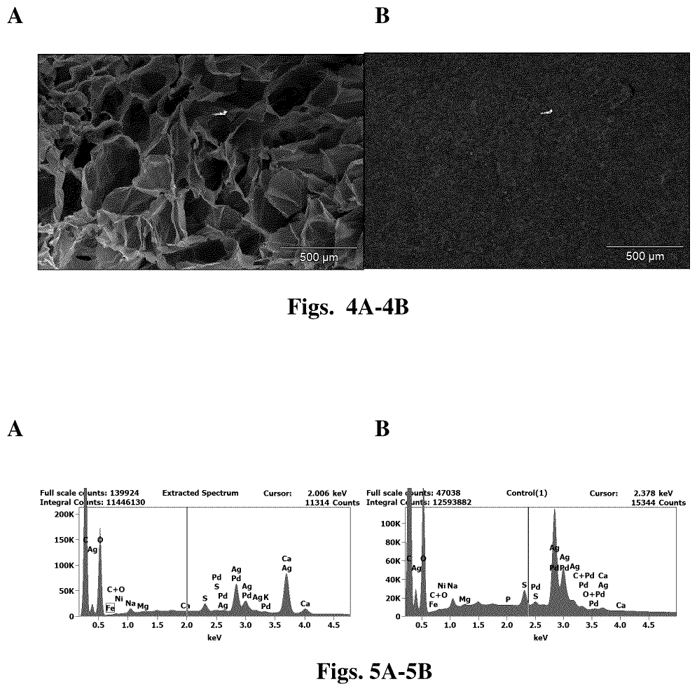

In some embodiments, the method further comprises mixing a calcium salt into the polymeric liquid solution. Examples of calcium salts include calcium chloride (CaCl₂) calcium lactate (CaC₆H₁₀O₆), calcium carbonate (CaCO₃), calcium citrate (Ca₃(C₆H₅O₇)₂), calcium phosphate (Ca₃(PO₄)₂), calcium acetate (CaC₄H₆O₄), calcium gluconate (CaC₆H₁₁O₇), CaCl₂H₂₂O₁₄, and CaCl₄H₂₆O₁₆. A preferred calcium salt for use in the method is calcium chloride (CaCl₂). Typically, the calcium salt is provided in a 1-2% aqueous solution, or in any range that results in a minimum Ca²⁺ ionic concentration of 0.018M in order to activate the coagulation cascade. FIG. 4A-4B illustrates that this embodiment of calcium addition allows the calcium to be properly distributed across the entire resulting scaffold.

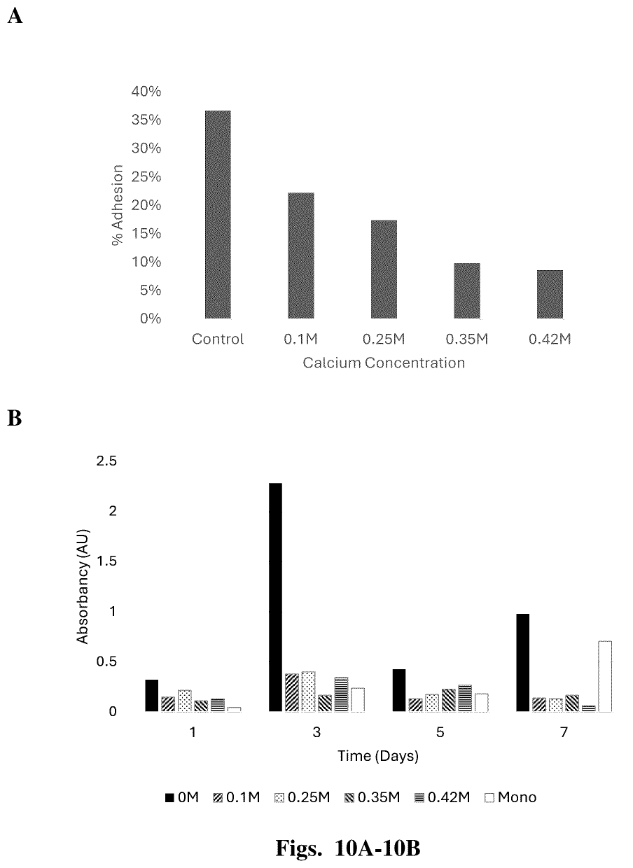

The inventors have observed that a calcium additive can affect cell activity. As shown in FIG. 10A-10B showing results of WST-1 assay (cell metabolic activity) ran over a period of 7 days on groups with increasing concentrations of calcium. Cells were seeded onto the scaffolds and left for one day, after which the scaffolds were taken out in order to determine percent adhesion. These results suggest differences in cell behavior depending on calcium concentrations.

The method includes mixing the gelatin, chitosan, and hyaluronate solutions to form a polymeric liquid solution. Mechanical agitation may be used to mix the gelatin, chitosan, and hyaluronate solutions in order to promote formation of the polymeric liquid solution. A polymeric liquid solution is one including polymer components (e.g., gelatin, chitosan, and hyaluronate) which has not yet formed a relatively solid polymer matrix. There is also an occasional requirement to achieve a relatively homogeneous suspension in a mixing vessel, particularly when this is being used to prepare materials for subsequent processes.

The method includes the step of pouring the polymeric liquid solution into a mold. The polymeric liquid solution can be poured and formed into a mold having any suitable shape or size for applying to a body part, including but not limited to disk/circles, ovals, rectangles, squares, kidney bean shape, or other contoured shapes. One illustrative embodiment uses a petri dish as a mold to form a thin disk shape. Another embodiment uses a tube-shaped mold to form a tampon-like shape for applications such as deep wounds.

The method includes mixing the gelatin, chitosan, and hyaluronate solutions to form a polymeric liquid solution. The gelatin, chitosan, and hyaluronate solutions be available for use as stock solutions, or they can be prepared immediately before use. Accordingly, in some embodiments, method steps can include preparing one or more of the gelatin solution, the high molecular weight chitosan solution, and the hyaluronate solution.

In some embodiments, the method provides a semi-synthetic scaffold having a polymer matrix comprising about 60-80 wt % gelatin, about 10-30 wt % chitosan having a molecular weight of 190 kDa or greater, and about 5-15 wt % hyaluronate. In additional embodiments, the gelatin, chitosan, and hyaluronate solutions are mixed in a about a 7-2-1 ratio.

The method also includes the step of gradually cooling the mixture to a temperature of about −80° C. for 12 to 24 hours to form a polymer matrix. Gradually cooling the mixture refers to cooling the material over a significant period of time, rather than suddenly cooling or quenching the mixture. For example, gradually cooling the mixture includes cooling the mixture for a period of about 12 to 24 hours in a refrigerator to an intermediate chilled temperature such as about 0° C. to about 5° C., and then further cooling the mixture to about −20° C. in a freezer for about 24 hours. Cooling to about −80° C. can be achieved using an ultrafreezer, which corresponds to freezing the polymer matrix.

The method also includes lyophilizing the polymer matrix to form a scaffold. Lyophilization is performed to remove water from the polymer matrix to form a dry polymer scaffold. Methods of lyophilization are well known to those skilled in the art. For example, lyophilization can be performed under vacuum that ranged from 0.600 mBar to 0.040 mBar, at a temperature of −50° C. or −80° C. for about 24 hours.

By incorporating a calcium salt (e.g. CaCl₂) into the polymer matrix the inventors found that they can use the Ge/Ch/HA matrix as a platform to help other compounds get delivered efficiently into tissue. For example, the hydrophobicity/hydrophilicity of different compounds affects their incorporation into the matrix, the role of pH and temperature in the process, and during which steps of the matrix preparation incorporate the different additives. This includes different calcium salts, nanoparticles (for antimicrobial or other purposes such as coagulation or cell growth), and biosensors (such as aptamers) in addition to autologous inserts known to those of skill in the art. Additionally considered is allogenic tissue (“off-the-shelf stem cells”) additions. Accordingly, in some embodiments, the method further includes adding one or more additives selected from the group consisting of an antimicrobial component, a coagulating component, a cosmetic component, a biologic component, a therapeutic component, and a diagnostic component to the polymeric liquid solution. These additives are preferably added to the liquid polymer solution before forming a polymer matrix.

### 7. SEMI-SYNTHETIC SCAFFOLDS

Another aspect of the invention provides a semi-synthetic scaffold. The semi-synthetic scaffold includes a polymer matrix having an average pore size from about 100 μm to about 250 μm, comprising about 60-80 wt % gelatin, about 10-30 wt % chitosan having a molecular weight of 190 kDa or greater, and about 5-15 wt % hyaluronate.

In some embodiments, the composition comprises about 65-75 wt % gelatin, about 15-25 wt % chitosan having a molecular weight of 190 kDa or greater, and about 7-12 wt % hyaluronate. In further embodiments, the composition comprises about 70 wt % gelatin, about 20 w % chitosan having a molecular weight of 190 kDa or greater, and about 10 wt % hyaluronate. In yet further embodiments, the present invention provides a semi-synthetic scaffold comprising gelatin, chitosan, and hyaluronic acid at the following ratios: 59-81% wt % gelatin (Ge) in water, 9-31% wt % high molecular weight chitosan (Ch), in water and acetic acid, and 5-15% wt % hyaluronic acid (HA) or sodium hyaluronate (SH) in water.

Cells, scaffolds, and growth-stimulating signals are generally referred to as the tissue engineering triad, the key components of engineered tissues. Scaffolds, typically made of polymeric biomaterials, provide the structural support for cell attachment and subsequent tissue development. A preferred scaffold for an engineered tissue should be the ECM of the target tissue in its native state. Nevertheless, the multiple functions, the complex composition and the dynamic nature of ECM in native tissues make it difficult to mimic exactly. Therefore, contemporary concepts of scaffolding in tissue engineering is to mimic the functions of native ECM, at least partially.

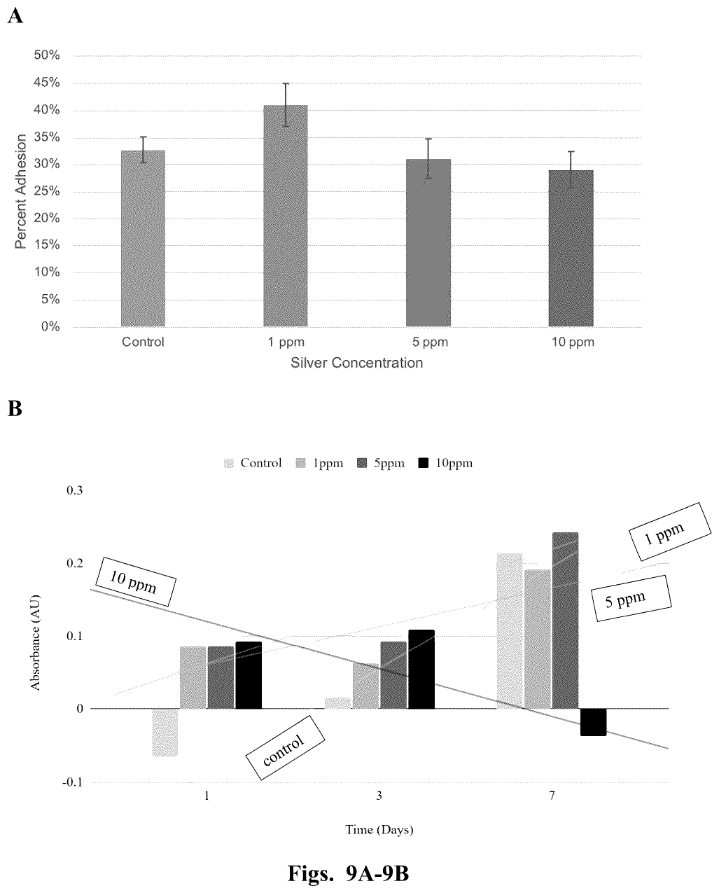

Scaffolds should provide void volume for vascularization, new tissue formation and remodeling so as to facilitate host tissue integration upon implantation. The polymer matrix should be processed to give a porous enough structure for efficient nutrient and metabolite transport without significantly compromising the mechanical stability of the scaffold. Moreover, the polymer matrix should also be degradable upon implantation at a rate matching that of the new matrix production by the developing tissue. Scaffolds provide mechanical and shape stability to the tissue defect. Scaffolds should also provide support for either extraneously applied or endogenous cells to attach, grow, and differentiate during both in vitro culture and in vivo implantation, and should interact with the cellular components of the engineered tissues actively to facilitate and regulate their activities. The biomaterials used to fabricate the scaffolds need to be compatible with the cellular components of the engineered tissues and endogenous cells in host tissue. The semi-synthetic scaffolds of the present invention provide all of these important features. The inventors have observed that the semi-synthetic scaffolds can provide excellent absorption/swelling capacity of the polymer matrix after 3 days was measured for various levels of additives vs. no additives as the control. This is illustrated in FIG. 8, showing the absorption capacity at various levels of calcium additive. Note that the control has very little weight, therefore it can absorb 40 times its weight. However, the samples absorb 20 times their weight, which is still remarkable swelling capacity but relatively start with more weight due to the scaffold structure. Additionally, they absorb less after 3 days because they deliver the additive, acting as a delivery system.

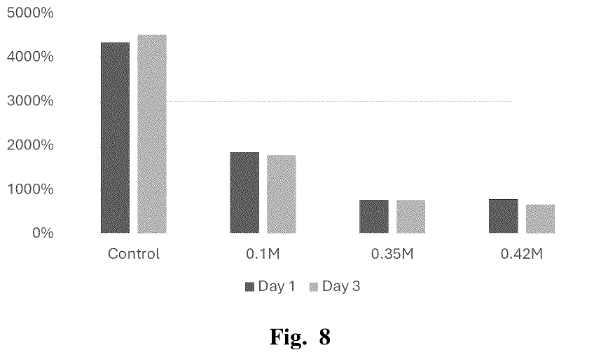

The semi-synthetic scaffold includes a polymer matrix. The term “polymer matrix,” as used herein, refers to a continuous phase of the components used to form the polymer matrix (e.g., gelatin, chitosan, and hyaluronate) that determines its properties and provides the capacity to retain other materials such as additives. The polymer matrix is a porous composition. In some embodiments, having an average pore size range from about 100 μm to about 250 μm, while in further embodiments the polymer matrix has an average pore size range from about 80 μm to about 300 μm, while in yet further embodiments the polymer matrix has an average pore size from about 125 μm to about 200 μm. As shown in FIG. 7, the addition of varying concentrations of additives does not appear to have a significant impact on the resulting average pore size of the matrix.

In some embodiments, the semi-synthetic scaffold includes a cross linker in the polymer matrix. Non-limiting examples of cross-linker agents include Genipin, 1-ethyl-3-(3-(dimethylaminopropyl)-carbodiimide (EDC), N-hydroxysuccinimide (NHS), and 2-morpholino-ethane sulfonic acid (MES).

The semi-synthetic scaffold includes a polymer matrix having about 60-80 wt % gelatin. gelatin sources include bovine, porcine, insectine, piscine, avian, plant-based, and human gelatin. Gelatin is a natural origin protein derived from collagen hydrolysis. Controlled release of bioactive molecules, formulations with conductive properties, or systems with improved mechanical properties can be obtained using gelatin composites. Many studies have found that the use of calcium phosphate ceramics and diverse synthetic polymers in combination with gelatin improve the mechanical properties of the structures. See Echave M C, et al., Gelatin as Biomaterial for Tissue Engineering. Curr Pharm Des., 23(24):3567-3584 (2017).

The semi-synthetic scaffold includes a polymer matrix having about 10-30 wt % chitosan. Chitosan is a natural polysaccharide with a positive charge, and is a derivative of chitin. It consists of the monomers N-acetyl-d-glucosamine and d-glucosamine linked by β-1,4-glycosidic bonds. Chitosan occurs naturally, and is typically obtained from chitin, which is one of the main components of the exoskeletons of arthropods such as shrimp, crabs, lobsters, and arachnids. The most common method for obtaining chitosan is the deacetylation of chitin, which is a two-stage nucleophilic substitution reaction that takes place in an alkaline solution. Depending on the origin of chitin and parameters of the deacetylation process, chitosan can be obtained with different molecular weights (MW) and degrees of deacetylation (DD). These parameters affect the physicochemical and biological properties of biopolymer-based materials. The properties of chitosan, such as biocompatibility, biodegradability, non-toxicity, antimicrobial activity, hemostatic and pain relief properties, and ability to accelerate wound healing, make it an object of research and application in designing dressing materials, scaffolds, and carriers for the controlled release of drugs and other biologically active pathways. Chitosan can also be used as a component of food packaging or as a food additive, inhibiting the development of microorganisms and thus prolonging the freshness of the product. One of the most significant features of the potential action of chitosan is the antimicrobial activity of the polymer, which is the result of many different factors, including DD and MW.

In some embodiments, the chitosan included in the polymer matrix has a molecular weight of 190 kDa or greater. The inventors have demonstrated that chitosan above 190 kDa can exhibit powerful antimicrobial and cellular properties and high adhesion. This phenomenon is probably correlated with the surface charge density distribution and amount of free amino groups. Chitosan having a higher MW results in greater amounts of and stronger electrostatic interactions between the chitosan chains, and thus the formation of a more developed polycation molecule that attaches more easily to the cell. This also improves shelf life and degradation time for cell scaffolding structures. In further embodiments, the chitosan has a MW above 150 kDa, above 165 kDa, above 180 kDa, above 200 kDa, above 225 kDa, or above 250 kDa. In embodiments where the Chitosan has lower molecular weight than 150 kDa, a plasticizer such as glycerol is used or a cross-linker alternative such as a calcium salt is incorporated.

The semi-synthetic scaffold includes a polymer matrix having about 5-15 wt % hyaluronate. Hyalorunate, as used herein, refers to both hyaluronic acid and its salts. Hyaluronic acid (HA) is a glycosaminoglycan constituted from two disaccharides (N-acetylglucosamine and D-glucuronic acid), isolated initially from the vitreous humor of the eye, and subsequently discovered in different tissues or fluids (especially in the articular cartilage and the synovial fluid). It is ubiquitous in vertebrates, including humans, and it is involved in diverse biological processes, such as cell differentiation, embryological development, inflammation, wound healing, etc.

Hyaluronic acid has a high molecular weight, acting as a macromolecule. The large molecules coat the skin and prevent water loss, leading to better hydration. Other, more “diluted” forms such as sodium hyaluronate (SH) have a lower concentration of HA as they are split by other salts or compounds, and therefore have a lower molecular weight than hyaluronic acid. SH is small enough to penetrate the epidermis, or top layer of the skin. In turn, it can improve hydration from the underlying skin layers. However, when compounded with the gelatin and chitosan structure, the sodium groups conflict with other cations such as calcium, making its incorporation vary according to clinical need. The inventors observed that, if incorporating SH, the calcium/coagulant groups must be incorporated in the final mixing step while preparing our composition. When incorporating pure HA, the calcium/coagulant groups must be incorporated in the initial step of mixing, specifically into the gelatin.

The inventors have found that when using SH, the calcium/coagulant groups are preferably incorporated in the final mixing step while preparing our composition. Alternately, when using pure HA, the calcium/coagulant groups are preferably incorporated in the initial step of mixing, specifically into the gelatin.

In some embodiments, the semi-synthetic scaffold includes one or more additives. Additives are additional compounds and/or components that confer additional properties and/or functions to the semi-synthetic scaffold. In some embodiments, the additives are selected from the group consisting of antimicrobial compounds, anti-inflammatory compounds, coagulating compounds, cosmetic compounds, biologic components, therapeutic components, and diagnostic components. The optional additives may be added to the liquid solution by mixing with one of the gelatin, chitosan, and hyaluronic acid solutions during the mixing steps.

In some embodiments, the semi-synthetic scaffold includes a calcium salt additive. This calcium salt additive can serve various functions to the scaffold based on desired physical properties and biologic properties, such as a hemostatic agent, or a cross-linker alternative. Suitable calcium salts include one or more of calcium chloride, calcium lactate, calcium carbonate, calcium phosphate, calcium acetate, and calcium gluconate.

In some embodiments the additive is an antimicrobial compound. Antimicrobial compounds can be used to help prevent infection in healing wounds. Examples of antimicrobial compounds include silver and silver salts, iodine and iodine salts, copper and copper salts, zinc and zinc salts, gold and gold salts, hypochlorous acid, sodium citrate, citric acid, chlorogenic acid, and benzalkonium chloride. Antimicrobial compounds also include antifungal, antibiotic, and antiviral compounds.

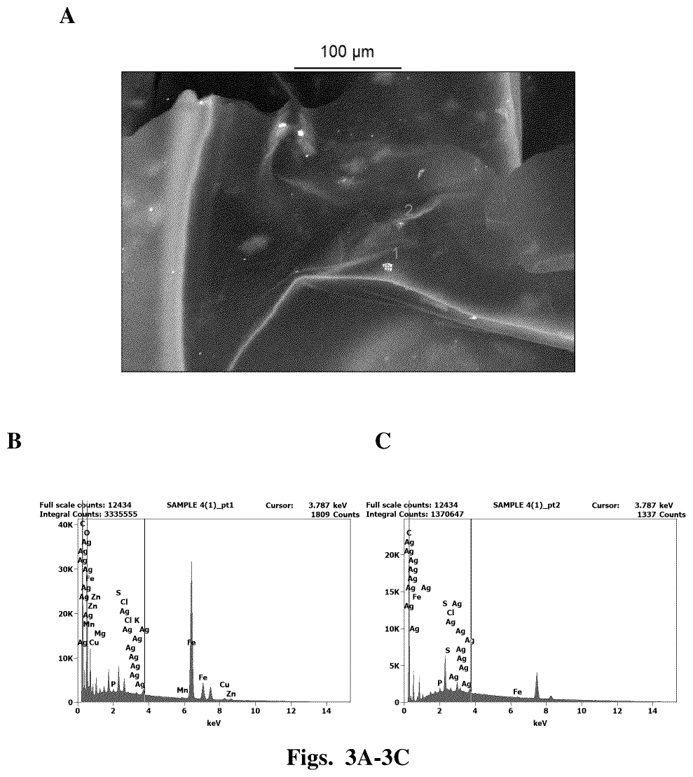

In some embodiments, the antimicrobial compound is a silver nanoparticle. The inventors have identified silver nanoparticles as a particularly promising antimicrobial additive known in the art. Testing has shown in FIGS. 3A-3C, and 5A-5B that such an additive can be successfully integrated into the semi-synthetic scaffold. Additionally, as shown in FIGS. 9A-9B, a silver additive can have a slight increase in percent cell adhesion. A WST-1 assay (cell metabolic activity) ran over a period of 7 days on groups with increasing concentrations of silver nanoparticles suggests the presence and concentration of silver nanoparticles has an impact on cell behavior. Cells were seeded onto the scaffolds and left for one day, after which the scaffolds were taken out in order to determine percent adhesion. There is a statistically significant improvement in cell adhesion in 1 ppm silver nanoparticles compared to no silver. Graph (B) shows absorbance over time, and trendlines for cellular activity. 1 and 5 ppm showed the best cell adhesion results. Furthermore, including such a small amount of silver nanoparticles allows for antimicrobial properties with minimal cost and downsides.

Various antibiotics can also be employed in the semi-synthetic scaffold, including, but not limited to: aminoglycosides, such as amikacin, gentamycin, kanamycin, neomycin, netilmicin, paromomycin, streptomycin, or tobramycin; carbapenems, such as ertapenem, imipenem, meropenem; chloramphenicol; fluoroquinolones, such as ciprofloxacin, gatifloxacin, gemifloxacin, grepafloxacin, levofloxacin, lomefloxacin, moxifloxacin, norfloxacin, ofloxacin, sparfloxacin, or trovafloxacin; glycopeptides, such as vancomycin; lincosamides, such as clindamycin; macrolides/ketolides, such as azithromycin, clarithromycin, dirithromycin, erythromycin, or telithromycin; cephalosporins, such as cefadroxil, cefazolin, cephalexin, cephalothin, cephapirin, cephradine, cefaclor, cefamandole, cefonicid, cefotetan, cefoxitin, cefprozil, cefuroxime, loracarbef, cefdinir, cefditoren, cefixime, cefoperazone, cefotaxime, cefpodoxime, ceftazidime, ceftibuten, ceftizoxime, ceftriaxone, or cefepime; monobactams, such as aztreonam; nitroimidazoles, such as metronidazole; oxazolidinones, such as linezolid; penicillins, such as amoxicillin, amoxicillin/clavulanate, ampicillin, ampicillin/sulbactam, bacampicillin, carbenicillin, cloxacillin, dicloxacillin, methicillin, mezlocillin, nafcillin, oxacillin, penicillin G, penicillin V, piperacillin, piperacillin/tazobactam, ticarcillin, or ticarcillin/clavulanate; streptogramins, such as quinupristin/dalfopristin; sulfonamide/folate antagonists, such as sulfamethoxazole/trimethoprim; tetracyclines, such as demeclocycline, doxycycline, minocycline, or tetracycline; azole antifungals, such as clotrimazole, fluconazole, itraconazole, ketoconazole, miconazole, or voriconazole; polyene antifungals, such as amphotericin B or nystatin; echinocandin antifungals, such as caspofungin or micafungin, or other antifungals, such as ciclopirox, flucytosine, griseofulvin, or terbinafine.

In some embodiments, the additive is an anti-inflammatory compound. Examples of anti-inflammatory compounds that can be incorporated into the semi-synthetic scaffold include, but are not limited to, steroidal anti-inflammatory agents such as betamethasone, triamcinolone dexamethasone, prednisone, mometasone, fluticasone, beclomethasone, flunisolide, and budesonide; and non-steroidal anti-inflammatory agents, such as fenoprofen, flurbiprofen, ibuprofen, ketoprofen, naproxen, oxaprozin, diclofenac, etodolac, indomethacin, ketorolac, nabumetone, sulindac tolmetin meclofenamate, mefenamic acid, piroxicam, and suprofen.

In some embodiments, the additive included in the semi-synthetic scaffold is a growth factor. Growth factors are endogenous signaling molecules that regulate cellular responses required for wound healing processes such as migration, proliferation, and differentiation. However, exogenous application of growth factors has limited effectiveness in clinical settings due to their low in vivo stability, restricted absorption through skin around wound lesions, elimination by exudation prior to reaching the wound area, and other unwanted side effects. See Park J W, Hwang S R, Yoon I S. Advanced Growth Factor Delivery Systems in Wound Management and Skin Regeneration. Molecules; 22(8):1259 (2017).

In some embodiments, the additive is a coagulant compound. Coagulant compounds can be used to speed up the natural clotting and coagulation process to facilitate wound healing. Examples of coagulants include calcium and calcium salts such as calcium carbonate, calcium chloride, calcium phosphate, clays, kaolin and kaolin salts, magnesium and magnesium salts, clays, bentonite, halloysite, zeolite, aluminosilicates, fibrinogen, prothrombins, thromboplastins, proaccelerins, proconvertins, vitamin K, and plasminogens.

In some embodiments, the additive is a salt. The nature of the salt is not critical, provided that it is biocompatible. Suitable acid addition salts of the compounds may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, γ-hydroxybutyric, galactaric, and galacturonic acids.

In some embodiments, the additive is a cosmetic compound. Cosmetic compounds can be used to reduce scarring, aging, wrinkling, hair growth, skin discoloration, or other qualities of cosmetic and visual value. Examples of such cosmetic compounds are chlorogenic acid, astaxanthin, Alpha Hydroxy Acids (AHAs), Aloe Vera, Antioxidants, Ascorbic Acid (Vitamin C), Bakuchiol, Biotin, Benzoyl Peroxide, Caffeine, Ceramides, Chamomile, Charcoal, Dimethicone, Dipotassium Glycyrrhizate, Echinacea, Emollients, Epidermal Growth Factors (EGF), Ferulic Acid, Ferrous Gluconate, Glycerin, Green Tea Extract, Hyaluronic Acid, Hemp Seed Oil, Illuminants, Jojoba Oil, Kojic Acid, Lactic Acid, Lavender Oil, Mandelic Acid, Milk Thistle, Niacinamide (Vitamin B3), Olive Oil, Omega Fatty Acids, Peptides, Phenoxyethanol, Polyhydroxy Acids (PHAs), Retinol (Vitamin A), Resveratrol, Salicylic Acid, Squalane, Sunscreen Ingredients, Tea Tree Oil, Tocopherol (Vitamin E), Witch Hazel, Zinc Oxide. In some embodiments, the additive is a biologic component. Biologics can be used for example for cell and blood therapy. Non-limiting embodiments of biologics include cells (e.g., stem cells) or blood-derived components, blood, platelet-rich plasma, and tissue.

In some embodiments, the semi-synthetic scaffold includes stem cells. Stem cells have two features: the ability to differentiate along different lineages and the ability of self-renewal. Two major types of stem cells have been described, namely, embryonic stem cells and adult stem cells. Embryonic stem cells (ESC) are obtained from the inner cell mass of the blastocyst and are associated with tumorigenesis, and the use of human ESCs involves ethical and legal considerations. The use of adult mesenchymal stem cells is less problematic with regard to these issues. Mesenchymal stem cells (MSCs) are stromal cells that have the ability to self-renew and also exhibit multilineage differentiation. MSCs can be isolated from a variety of tissues, such as umbilical cord, endometrial polyps, menses blood, bone marrow, adipose tissue, etc.

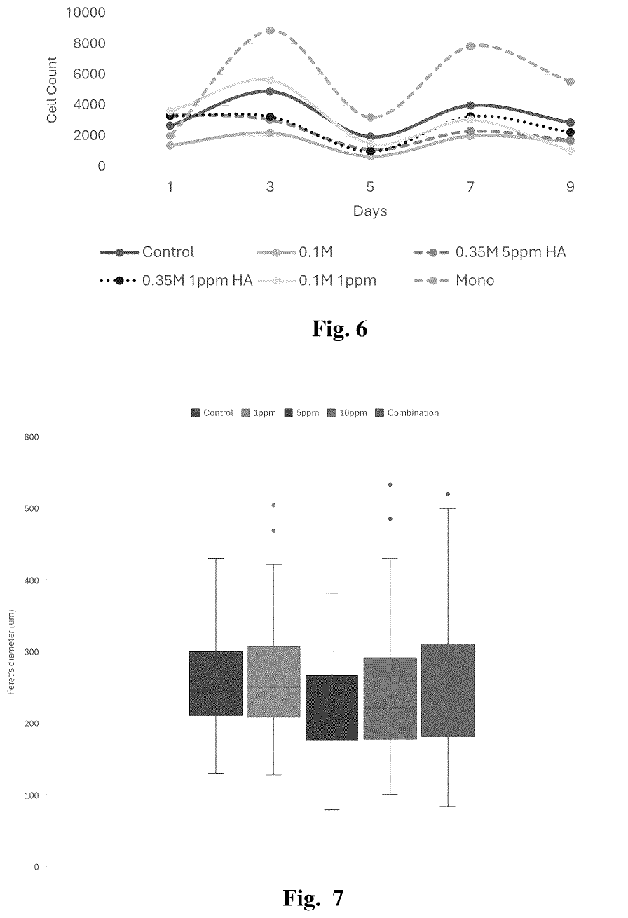

In some embodiments, the additive is a diagnostic component. Examples of diagnostic components include sensors, microchips, aptamer-based sensors and biosystems, antibody-based sensing systems, and microcontrollers. The inventors have observed addition of aptamers using luciferase-expressing cells in order to quantify the number of cells present in a dish with different scaffold groups present. Through this method we observed the behaviors of cells that we can modulate through the use of showing different additives. This is illustrated in FIG. 6.

In some embodiments, the additive can be included in a nanoparticle or other nano-encapsulation methods. Nanoparticles are submicron materials that often possess different properties than bulk material of the same kind. Nanoparticles have been studied for uses in many fields, including diagnostic and therapeutic applications in life sciences. Because of their small size and unique properties, nanoparticles often have enhanced distribution in the body compared to larger sized particles. Further, nanoparticles may be specifically directed to particular targets in the body by attaching one or more components to the nanoparticle surface (i.e. functionalization).

Nanoparticles, as the term is used herein, are particles having a matrix-type structure with a size of 1000 nanometers or less. The nanoparticles are generally spherical structures. In some embodiments, the nanoparticles have a size of 500 nanometers or less. In some embodiments, the particles have a diameter from 10 nanometers to 1000 nanometers. In other embodiments, the particles have a diameter from 10 nanometers to 500 nanometers. In further embodiments, the particles have a diameter from 10 to 300 nanometers, while in yet further embodiments the particles have a diameter from 50 to 300 nanometers. The diameter of the nanoparticles refers to their mean hydrodynamic diameter. The hydrodynamic diameter can be readily determined using dynamic light scattering (DLS).

The nanoparticles of the invention can be prepared using a wide variety of different types of polymers. Preferably, the nanoparticle comprises one or more biocompatible polymers. Examples of biocompatible polymers include natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like.

In some embodiments, the bioactive flavonoid of the Epimedium plant, Icariin (ICRN) is included as an additive. ICRN has a broad range of applications in improving scaffolds as a constant and non-immunogenic material, and in stimulating the cell growth, differentiation of chondrocytes as well as differentiation of embryonic stem cells towards cardiomyocytes. Fusion of ICRN into scaffolds can enhance the secretion of the collagen matrix and proteoglycan in bone and cartilage tissue engineering. In addition, ICRN can induce apoptosis, reduce viability and inhibit proliferation of cancer cells, and repress tumorigenesis as well as metastasis. Moreover, cancer cells no longer grow by halting the cell cycle at two checkpoints, G0/G1 and G2/M, through the inhibition of NF-κB by ICRN.

The semi-synthetic scaffold can be prepared using the methods described herein. In one embodiment, the scaffold is prepared by mixing a gelatin solution, a high molecular weight chitosan solution, and a hyaluronate solution, to form a polymeric liquid solution; pouring the polymeric liquid solution into a mold; gradually cooling the mixture to a temperature of −80° C. for 12 to 24 hours to form a polymer matrix; and freezing and lyophilizing the polymer matrix to form a scaffold.

A tissue scaffold is a support structure that provides a matrix for cells to guide the process of tissue formation during wound healing or in vivo. The size, shape and geometry of scaffold may vary depending on the intended use. A membrane like scaffold with size ranging from 1 square inch to 1000 square inch may be used for large scale manufacturing of membrane-like tissue. The scaffold could also be circular, rectangular, triangular, hexagonal or any other 2-dimensional shape. Several membrane-like scaffolds may be stacked in a biotechnology tissue engineering reactor to generate large quantity of engineered tissues suitable for large-scale bioprosthesis manufacturing. The scaffold could also have simple or complex three-dimensional shapes or geometries. These geometries may include but not limited are: hollow cylindrical tube, cylinder, sphere or complex shapes. Membrane shaped tissue engineered tissue may also be created by using a hollow cylindrical tube-like scaffold and then cutting the tube-shaped engineered tissue to make a sheet or membrane like tissue.

Typically, the scaffold is configured to have the shape of the tissue that it is being substituted for. However, the scaffold material can also be used for cosmetic work or “bioengineering,” where a support structure is provided for the creation of new tissue rather than the replacement or regeneration of existing tissue.

In some embodiments, the scaffold may formed into a dry solid matrix, a wet, viscous hydrogel form, a foam form, a dry powder, a dry sheet, an aerogel, or encapsulated in hardware as a wearable device.

The semi-synthetic scaffold is made of a biodegradable polymer matrix, and hence is bioresorbable. Bioresorbable, as used herein, refers to the ability of the scaffolds to be gradually degraded by physiological processes in vivo, to allow the replacement of the scaffold with native tissue. For example, if the scaffold is used to replace endothelial tissue, the scaffold may be gradually degraded while endothelial cells rebuild endothelial tissue in its place.

### 8. WOUND HEALING USING SEMI-SYNTHETIC SCAFFOLDS

Another aspect of the invention provides a method of wound healing. The method includes contacting a wound of a subject with a semi-synthetic scaffold, wherein the semi-synthetic scaffold comprises a polymer matrix having an average pore size from about 100 μm to about 250 μm, comprising about 60-80 wt % gelatin, about 10-30 wt % chitosan having a molecular weight of 190 kDa or greater, and about 5-15 wt % hyaluronate. The semi-synthetic scaffold used in the method can be any of the semi-synthetic scaffolds described herein. For example, in some embodiments, the semi-synthetic scaffold further comprises one or more additives selected from the group consisting of an antimicrobial component, a coagulating component, a cosmetic component, a biologic component, a therapeutic component, and a diagnostic component.

As used herein, the term “wound healing” refers to a regenerative process with the induction of an exact temporal and spatial healing program comprising wound closure and the processes involved in wound closure. The term “wound healing” encompasses but is not limited to the processes of granulation, neovascularization, fibroblast, endothelial and epithelial cell migration, extracellular matrix deposition, re-epithelialization, and remodeling.

In some embodiments, the present invention provides methods for “accelerating wound healing,” whereby different aspects of the wound healing process are “enhanced.” As used herein, the term “enhanced” indicates that the methods provide an increased rate of wound healing. In preferred embodiments, the term “enhanced” indicates that the wound healing rate and/or a wound healing process occurs at least 10% faster than is observed in untreated or control-treated wounds. In particularly preferred embodiments, the term “enhanced” indicates that the wound healing rate and/or a wound healing process occurs at least 15% faster than is observed in untreated or control-treated wounds. In still further preferred embodiments, the term “enhanced” indicates that the wound healing rate and/or a wound healing process occurs at least 20% (e.g., 50%, 100%, . . . ) faster than wounds untreated or control-treated wounds.

Contacting, as used herein, refers to causing two items to become physically adjacent and in contact, or placing them in an environment where such contact will occur within a reasonably short timeframe. For example, contacting a site with a semi-synthetic scaffold includes placing the scaffold over all or a portion of the wound site.

As used herein, the term “wound” refers to a disruption of the normal continuity of structures caused by a physical (e.g., mechanical) force, a biological (e.g., thermic or actinic force, or a chemical means. In particular, the term “wound” encompasses wounds of the skin. The term “wound” also encompasses contused wounds, as well as incised, stab, lacerated, open, penetrating, puncture, abrasions, grazes, burns, frostbites, corrosions, wounds caused by ripping, scratching, pressure, and biting, and other types of wounds.

In some embodiments, the skin wound being treated is a chronic wound. As used herein, the term “chronic wound” refers to a wound that does not fully heal even after a prolonged period of time (e.g., 2 to 3 months or longer). The treatment of chronic infections of the skin often is a challenge to clinicians. Infected, burns, surgical wounds, and diabetic lesions can be refractory to current treatment regimens causing them to persist as open sores. Chronic wounds often occur in patients having impaired wound healing. Impaired wound healing can be a result of a variety of conditions, such as infection of the wound, the patient being diabetic, or the patient being elderly.

As described herein, calcium can be included during the preparation of the semi-synthetic scaffold. Apart from being a critical coagulation factor during hemostasis, the calcium ion has also been shown to act as a fundamental cue, directing the cellular functions of different types of cells during wound healing. Calcium plays a vital role as the extracellular signaling molecule and intracellular second messenger for keratinocytes and fibroblasts. Previous studies have explored the impact of calcium concentrations on keratinocyte proliferation and differentiation. However, the effects of calcium on dermal fibroblasts have yet to be fully elucidated. A modest number of studies have shown that calcium influences the morphology, proliferation, and collagen deposition of fibroblasts. Subramaniam T, et al., The Role of Calcium in Wound Healing. Int J Mol Sci., 22(12):6486 (2021).

Wound healing is a natural physiological reaction to tissue injury. However, wound healing is not a simple phenomenon but involves a complex interplay between numerous cell types, cytokines, mediators, and the vascular system. The cascade of initial vasoconstriction of blood vessels and platelet aggregation is designed to stop bleeding. This is followed by an influx of a variety of inflammatory cells, starting with the neutrophil. These inflammatory cells, in turn, release a variety of mediators and cytokines to promote angiogenesis, thrombosis, and re-epithelialization. The fibroblasts, in turn, lay down extracellular components which will serve as scaffolding. See Wallace H A, Basehore B M, Zito P M. Wound Healing Phases, Treasure Island (FL): StatPearls Publishing, 2023.

### 9. BIOMANUFACTURING AND GUIDED TISSUE REGENERATION USING SEMI-SYNTHETIC SCAFFOLDS

Guided tissue regeneration (GTR) and organ biomanufacturing are pivotal advancements in regenerative medicine. GTR involves the use of barrier membranes to direct the growth of new bone and soft tissues at sites where they are deficient, essentially creating an environment where the body's natural healing processes can flourish. The method includes incorporating a semi-synthetic scaffold into a bioreactor in a laboratory setting to create tissue artificially in a biomanufacturing or laboratory environment, wherein the semi-synthetic scaffold comprises a polymer matrix having an average pore size from about 100 μm to about 250 μm, comprising about 60-80 wt % gelatin, about 10-30 wt % chitosan having a molecular weight of 190 kDa or greater, and about 5-15 wt % hyaluronate. The semi-synthetic scaffold used in the method can be any of the semi-synthetic scaffolds described herein. For example, in some embodiments, the semi-synthetic scaffold further comprises one or more additives selected from the group consisting of an antimicrobial component, a coagulating component, a cosmetic component, a biologic component, a therapeutic component, and a diagnostic component.

As used herein, the term “Biomanufacturing” refers to a regenerative process with the induction of an exact temporal and spatial cellular growth program comprising tissue formation and the processes involved in tissue formation. The term “tissue formation” encompasses but is not limited to the processes of granulation, neovascularization, fibroblast, endothelial and epithelial cell migration, extracellular matrix deposition, re-epithelialization, and remodeling in an artificial laboratory setting.

In some embodiments, the organ being manufactured is being expanded from host tissue. As used herein, the term “host tissue” refers to an autologous or heterologous sample that is expanded in a laboratory or biomanufacturing setting. The lack of scalable biomanufacturing is a significant problem for clinicians because it limits the availability of advanced therapies and treatments. Without the ability to produce biological products like cell-based therapies, tissue-engineered organs, and regenerative medicine products on a large scale, these innovative treatments remain inaccessible to many patients who need them. This scarcity can lead to longer wait times for treatment, higher costs, and reduced overall effectiveness of healthcare systems.

This method is particularly impactful in periodontal and orthopedic applications, where precise and localized tissue formation is essential. Organ biomanufacturing takes tissue engineering a step further, utilizing techniques like 3D bioprinting and scaffold-based approaches to fabricate complex, functional organs. By layering cells, growth factors, and biomaterials in a highly controlled manner, researchers are making strides toward producing organs that can be used for transplantation. This innovation not only aims to address the critical shortage of donor organs but also reduces the risk of rejection by using the patient's own cells.

In order that the subject matter disclosed herein may be more efficiently understood, an example is provided below. It should be understood that this example is for illustrative purposes only and is not to be construed as limiting the claimed subject matter in any manner.

### 10. EXAMPLES

### 11. Example 1—“Base Compound”

### 1. Solution Preparation

2% Chitosan Solution (MW above 190 kDa)

A 2% w/v high molecular weight chitosan solution was prepared. This was achieved by adding acetic acid in a 1:100 ratio to water in order to obtain a 1%-1.5% by volume solution. After this, a 2:100 part of chitosan was added to the solution, obtaining a 2% w/v chitosan solution. This solution was stirred overnight at a temperature of 55° C. (131° F.).

1% Gelatin Solution

A 1% w/v type B gelatin (bovine or porcine origin) solution was prepared. This was achieved by adding type B gelatin powder in a 1:100 ratio to water. The solution was stirred at 55° C. (131° F.) until the gelatin was completely dissolved.

0.01% Hyaluronic Acid or Sodium Hyaluronate Solution

A 0.01% w/v sodium hyaluronate solution was prepared. This was achieved by adding a 1.6% w/v sodium hyaluronate solution in a 0.64:100 ratio to water in order to obtain a 0.01% w/v sodium hyaluronate solution. This solution was then heated up to 35° C. (95° F.).

### 2. Mixing

The gelatin solution was mixed with the Chitosan solution in a 7:2 weight ratio and left to agitate for 3 hours. This stage is optimal for the addition of different bioactive compounds. Once the 3 hours passed, the Sodium Hyaluronate Solution was added in a 1:9 weight ratio to the Gelatin chitosan solution, to end up having approximately a 7:2:1 weight ratio of Gelatin and Calcium and Chitosan and Sodium Hyaluronate solutions respectively. This was conducted at a necessary pH of 7.2. The end result of this was the base liquid polymeric solution.

### 3. Pouring

Once the base polymeric solution was prepared, this was poured into 80 mm petri dishes. 20 mL of the solution was poured into each petri dish to achieve a thickness of 3 mm.

### 4. Gradual Cooling

The petri dishes with the base polymeric solution were cooled in a refrigerator at 4° C. (39.2° F.) for at least 12 hours but no more than 24 hours. After this, the petri dishes with the base polymeric solution were frozen in a −20° C. (−4° F.) freezer for 24 hours. After this, the cooler with the petri dishes was transferred to an ultrafreezer at −80° C. (−112° F.) for at least 24 hours.

### 5. Lyophilization (Freeze Drying)

The petri dishes were removed from the ultrafreezer and put inside a lyophilizer. The lyophilization was performed under vacuum that ranged from 0.600 mBar to 0.040 mBar, at a temperature of −50° C. or −80° C. and for a duration of 24 hours. This removed the water from the samples and resulted in a dry polymer.

“Base compound” comprises 59-81% wt % gelatin, 9-31% wt % chitosan having a molecular weight of 190 kDa or greater, and 5-15% wt % hyaluronic acid or sodium hyaluronate. These ranges have been found to provide a sturdy scaffold matrix.

### 12. Example 2—Calcium Additive

### 13. Solution Preparation

2% Chitosan Solution

A 2% w/v high molecular weight chitosan solution was prepared. This was achieved by adding acetic acid in a 1:100 ratio to water in order to obtain a 1%-1.5%/v solution. After this, a 2:100 part of chitosan was added to the solution, obtaining a 2% w/v chitosan solution. This solution was stirred overnight at a temperature of 55° C. (131° F.).

1.1% CaCl₂ Solution

A 1.1% w/v CaCl₂ Solution was prepared. This was achieved by adding CaCl₂ in a 1.1:100 ratio to the HEPES Solution mentioned before. This was mixed until the salt was completely dissolved.

1% Gelatin Solution+Cross-Linking/Functionalization

A 1% w/v type B gelatin (bovine origin) solution was prepared. This was achieved by adding type B gelatin powder in a 1:100 ratio to water. The solution was stirred at 55° C. (131° F.) until the gelatin was completely dissolved. 20 Ml of the CaCl₂ Solution was added to the gelatin solution and left to react for 3 hours. This step has shown to strengthen the intrinsic structure of the polymer.

0.01% Sodium Hyaluronate Solution

A 0.01% w/v sodium hyaluronate solution was prepared. This was achieved by adding a 1.6% w/v sodium hyaluronate solution in a 0.64:100 ratio to water in order to obtain a 0.01% w/v sodium hyaluronate solution. This Solution was then heated up to 35° C. (95° F.)

### 14. Mixing

The gelatin solution was mixed with the Chitosan solution in a 7:2 ratio and left to agitate for 3 hours. This stage is optimal for the addition of different bioactive compounds. Once the 3 hours passed, the Sodium Hyaluronate Solution was added in a 1:9 ratio to the Gelatin chitosan solution, to end up having a 7:2:1 ratio of Gelatin and Calcium and Chitosan and Sodium Hyaluronate solutions respectively. This was conducted at a necessary pH of 7.2. The end result of this was the base liquid polymeric solution.

### 15. Pouring

Once the final solution was prepared, this was poured into 80 mm petri dishes. 20 mL of the solution was poured into each petri dish to achieve a thickness of 3 mm.

### 16. Gradual Cooling

The petri dishes with the base polymeric solution were cooled in a refrigerator at 4° C. (39.2° F.) for at least 12 hours but no more than 24 hours. The petri dishes with the base polymeric solution were frozen in a −20° C.(−4° F.) freezer for 24 hours. After this, the cooler with the petri dishes was transferred to an ultrafreezer at −80° C. (−112° F.) for at least 12 hours.

### 17. Lyophilization (Freeze Drying)

The petri dishes were removed from the ultrafreezer and put inside a lyophilizer. The lyophilization was performed under vacuum that ranged from 0.600 mBar to 0.040 mBar, at a temperature of −50° C. and for a duration of 24 hours. This removed the water from the samples and resulted in a dry polymer.

### 18. Example 3—Copper Addition

### 1. Solution Preparation

2% chitosan Solution+copper functionalization

A 2% w/v high molecular weight chitosan solution was prepared. This was achieved by adding acetic acid in a 1:100 ratio to water in order to obtain a 1%-1.5%/v solution. After this, a 2:100 part of chitosan was added to the solution, obtaining a 2% w/v chitosan solution. This solution was stirred overnight at a temperature of 55° C. (131° F.). 20 ML of the CaClₕ Solution was added to the gelatin solution and left to react for 3 hours. This step has shown to strengthen the intrinsic structure of the polymer

1.1% Copper Solution

A 1.1% w/v copper solution was prepared. This was achieved by adding copper powder in a 1.1:100 ratio to a phosphate-buffered saline solution. This was mixed until the copper was completely dissolved.

1% Gelatin Solution

A 1% w/v type B gelatin (bovine origin) solution was prepared. This was achieved by adding type B gelatin powder in a 1:100 ratio to water. The solution was stirred at 55° C. (131° F.) until the gelatin was completely dissolved.

0.01% Sodium Hyaluronate Solution

A 0.01% w/v Sodium Hyaluronate solution was prepared. This was achieved by adding a 1.6% w/v Sodium Hyaluronate solution in a 0.64:100 ratio to water in order to obtain a 0.01% w/v Sodium Hyaluronate solution. This Solution was then heated up to 35° C. (95° F.)

### 2. Mixing

The gelatin solution was mixed with the Chitosan solution in a 7:2 ratio and left to agitate for 3 hours. This stage is optimal for the addition of different bioactive compounds. Once the 3 hours passed, the Sodium Hyaluronate Solution was added in a 1:9 ratio to the Gelatin chitosan solution, to end up having a 7:2:1 ratio of Gelatin and Calcium and Chitosan and Sodium Hyaluronate solutions respectively. This was conducted at a necessary pH of 7.2. The end result of this was the base liquid polymeric solution.

### 3. Pouring

Once the final solution was prepared, this was poured into 80 mm petri dishes. 20 mL of the solution was poured into each petri dish to achieve a thickness of 3 mm.

### 4. Gradual Cooling

The petri dishes with the base polymeric solution were cooled in a refrigerator at 4° C. (39.2° F.) for at least 12 hours but no more than 24 hours. After this was done, the petri dishes with the base polymeric solution were frozen in a −20° C. (−4° F.) freezer for 24 hours. After this, the cooler with the petri dishes was transferred to an ultrafreezer at −80° C. (−112° F.) for at least 12 hours.

### 5. Lyophilization (Freeze Drying)

The petri dishes were removed from the ultrafreezer and put inside a lyophilizer. The lyophilization was performed under vacuum that ranged from 0.600 mBar to 0.040 mBar, at a temperature of −50° C. and for a duration of 24 hours. This removed the water from the samples and resulted in a dry polymer.

### 19. Example 4—Chlorogenic Acid Additive

### 1. Solution Preparation

2% Chitosan Solution

A 2% w/v high molecular weight Chitosan solution was prepared. This was achieved by adding acetic acid in a 1:100 ratio to water in order to obtain a 1%-1.5%/v solution. After this, a 2:100 part of chitosan was added to the solution, obtaining a 2% w/v chitosan solution. This solution was stirred overnight at a temperature of 55° C. (131° F.).

1.1% CGA Solution

A 1.1% w/v Chlorogenic Acid (CGA) Solution was prepared. This was achieved by adding CGA in a 1.1:100 ratio to water. This was mixed until the salt was completely dissolved.

1% Gelatin Solution+Cross-Linking/Functionalization

A 1% w/v type B gelatin (bovine origin) solution was prepared. This was achieved by adding type B gelatin powder in a 1:100 ratio to water. The solution was stirred at 55° C. (131° F.) until the gelatin was completely dissolved.

0.01% Sodium Hyaluronate Solution

A 0.01% w/v Sodium Hyaluronate solution was prepared. This was achieved by adding a 1.6% w/v Sodium Hyaluronate solution in a 0.64:100 ratio to water in order to obtain a 0.01% w/v Sodium Hyaluronate solution. This Solution was then heated up to 35° C. (95° F.)

### 2. Mixing

The gelatin solution was mixed with the Chitosan solution in a 7:2 ratio and left to agitate for 3 hours. This stage is optimal for the addition of different bioactive compounds. Once the 3 hours passed, the Sodium Hyaluronate Solution was added in a 1:9 ratio to the Gelatin chitosan solution, to end up having a 7:2:1 ratio of Gelatin and Calcium and Chitosan and Sodium Hyaluronate solutions respectively. 20 mL of the CGA Solution was added to the Gelatin solution and left to react for 3 hours. This step has shown to strengthen the intrinsic structure of the polymer. This was conducted at a necessary pH of 7.2. The end result of this was the base liquid polymeric solution.

### 3. Pouring

Once the final solution was prepared, this was poured into 80 mm petri dishes. 20 mL of the solution was poured into each petri dish to achieve a thickness of 3 mm.

### 4. Gradual Cooling

The petri dishes with the base polymeric solution were cooled in a refrigerator at 4° C. (39.2° F.) for at least 12 hours but no more than 24 hours. After this was done, the petri dishes with the base polymeric solution were frozen in a −20° C. (−4° F.) freezer for 24 hours. After this, the cooler with the petri dishes was transferred to an Ultrafreezer at −80° C. (−112° F.) for at least 12 hours.

### 5. Lyophilization (Freeze Drying)

The petri dishes were removed from the ultrafreezer and put inside a Lyophilizer. The lyophilization was performed under vacuum that ranged from 0.600 mBar to 0.040 mBar, at a temperature of −50° C. and for a duration of 24 hours. This removed the water from the samples and resulted in a dry polymer.

### 20. Example 5—Method Using Calcium Lactate

### 21. 1. Solution Preparation

2% Chitosan Solution

A 2% w/v high molecular weight Chitosan solution was prepared. This was achieved by adding acetic acid in a 1:100 ratio to water in order to obtain a 1%-1.5%/v solution. After this, a 2:100 part of chitosan was added to the solution, obtaining a 2% w/v chitosan solution. This solution was stirred overnight at a temperature of 55° C. (131° F.).

1.1% C₆H₁₀CaO₆ Solution

A 1.1% w/v C₆H₁₀CaO₆ (Calcium Lactate) solution was prepared. This was achieved by adding C₆H₁₀CaO₆ in a 1.1:100 ratio to water. This was mixed until the salt was completely dissolved.

1% Gelatin Solution+Cross-Linking/Functionalization

A 1% w/v type B gelatin solution was prepared. This was achieved by adding type B gelatin powder in a 1:100 ratio to water. The solution was stirred at 55° C. (131° F.) until the gelatin was completely dissolved. 20 mL of the C₆H₁₀CaO₆ solution (or any Calcium solution) was added to the Gelatin solution and left to react for 3 hours. This step has shown to strengthen the intrinsic structure of the polymer.

0.01% Sodium Hyaluronate Solution

A 0.01% w/v Sodium Hyaluronate solution was prepared. This was achieved by adding a 1.6% w/v Sodium Hyaluronate solution in a 0.64:100 ratio to water in order to obtain a 0.01% w/v Sodium Hyaluronate solution. This Solution was then heated up to 35° C. (95° F.)

### 22. 2. Mixing

The gelatin solution was mixed with the Chitosan solution in a 7:2 ratio and left to agitate for 3 hours. This stage is optimal for the addition of different bioactive compounds. Once the 3 hours passed, the Sodium Hyaluronate Solution was added in a 1:9 ratio to the Gelatin chitosan solution, to end up having a 7:2:1 ratio of Gelatin and Calcium and Chitosan and Sodium Hyaluronate solutions respectively. This was conducted at a necessary pH of 7.2. The end result of this was the base liquid polymeric solution.

### 23. 3. Pouring

Once the final solution was prepared, this was poured into 80 mm petri dishes. 20 mL of the solution was poured into each petri dish to achieve a thickness of 3 mm.

### 24. 4. Gradual Cooling

The petri dishes with the base polymeric solution were cooled in a refrigerator at 4° C. (39.2° F.) for at least 12 hours but no more than 24 hours. After this, the petri dishes with the base polymeric solution were frozen in a −20° C. (−4° F.) freezer for 24 hours. After this, the cooler with the petri dishes was transferred to an Ultrafreezer at −80° C. (−112° F.) for at least 24 hours.

### 25. 5. Lyophilization (Freeze Drying)

The petri dishes were removed from the ultrafreezer and put inside a Lyophilizer. The lyophilization was performed under vacuum that ranged from 0.600 mBar to 0.040 mBar, at a temperature of −50° C. OR −80° C. and lasted 24 hours. This removed the water from the samples and resulted in a dry polymer.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of making a semi-synthetic scaffold comprising:
mixing a gelatin solution, a high molecular weight chitosan solution, and a hyaluronate solution, to form a polymeric liquid solution;
pouring the polymeric liquid solution into a mold;
gradually cooling the mixture to form a polymer matrix; and
freezing and lyophilizing the polymer matrix to form a scaffold.

2. The method of claim 1, further comprising adding one or more additives selected from the group consisting of an antimicrobial component, a coagulating component, a cosmetic component, a biologic component, a therapeutic component, and a diagnostic component to the polymeric liquid solution.

3. The method of claim 1, further comprising mixing a calcium salt into the polymeric liquid solution.

4. The method of claim 3, wherein the calcium salt is one or more selected from the group consisting of calcium chloride, calcium lactate, calcium carbonate, calcium phosphate, calcium citrate, calcium acetate, and calcium gluconate.

5. The method of claim 1, further comprising preparing one or more of the gelatin solution, the high molecular weight chitosan solution, and the hyaluronate solution.

6. The method of claim 1, wherein the polymer matrix comprises about 60-80 wt % gelatin, about 10-30 wt % chitosan having a molecular weight of 190 kDa or greater, and about 5-15 wt % hyaluronate.

7. The method of claim 1, wherein the steps of freezing and lyophilizing the polymer matrix are only done once.

8. The method of claim 1, wherein a crosslinking agent is not added after freezing and lyophilizing the polymer matrix.

9. A semi-synthetic scaffold comprising a polymer matrix having an average pore size from about 100 μm to about 250 μm, comprising
about 60-80 wt % gelatin,
about 10-30 wt % chitosan having a molecular weight of 190 kDa or greater, and
about 5-15 wt % hyaluronate.

10. The semi-synthetic scaffold of claim 9, further comprising an antimicrobial compound.

11. The semi-synthetic scaffold of claim 9, further comprising a coagulating compound.

12. The semi-synthetic scaffold of claim 9, further comprising a diagnostic component.

13. The semi-synthetic scaffold of claim 9, further comprising a biologic component.

14. The semi-synthetic scaffold of claim 9, wherein the composition comprises about 65-75 wt % gelatin, about 15-25 wt % chitosan having a molecular weight of 190 kDa or greater, and about 7-12 wt % hyaluronate.

15. The semi-synthetic scaffold of claim 9, wherein the scaffold is prepared by mixing a gelatin solution, a high molecular weight chitosan solution, and a hyaluronate solution, to form a polymeric liquid solution; pouring the polymeric liquid solution into a mold; gradually cooling the mixture to form a polymer matrix; and freezing and lyophilizing the polymer matrix to form a scaffold.

16. A method of wound healing, comprising contacting a wound of a subject with a semi-synthetic scaffold, wherein the semi-synthetic scaffold comprises a polymer matrix having an average pore size from about 100 μm to about 250 μm, comprising about 60-80 wt % gelatin, about 10-30 wt % chitosan having a molecular weight of 190 kDa or greater, and about 5-15 wt % hyaluronate.

17. The method of claim 16, wherein the semi-synthetic scaffold further comprises one or more additives selected from the group consisting of an antimicrobial compound, a coagulating compound, a cosmetic compound, a biologic component, a therapeutic component, and a diagnostic component.

18. The method of claim 16, wherein the wound is a skin wound.

19. The method of claim 16, wherein the subject is a human.
